(19) United States
(12) Patent Application Publication
(10) Pub. No.: US 2023/0372155 A1
(43) Pub. Date: Nov. 23, 2023

(54) METHOD FOR PROVIDING CONTROL DATA FOR A LASER OF A TREATMENT APPARATUS

(71) Applicant: SCHWIND eye-tech-solutions GmbH, Kleinostheim (DE)

(72) Inventors: Samuel ARBA MOSQUERA, Aschaffenburg (DE); Tobias KEHRER, Kleinostheim (DE)

(21) Appl. No.: 18/315,195

(22) Filed: May 10, 2023

(30) Foreign Application Priority Data
May 17, 2022 (DE) 10 2022 112 296.4

(51) Int. Cl. A61F 9/008

(52) U.S. Cl. CPC A61F 9/00827; A61F 9/00804; A61F 2009/00872

(57) ABSTRACT

The invention relates to a method for providing control data for a laser (18) of a treatment apparatus (10) for the correction of a cornea (26), including ascertaining (S10) an effect of a deformation of the cornea (26) on preset corneal parameters by means of a corneal deformation model, wherein the cornea (26) can be modeled in a deformed and non-deformed state by the corneal deformation model, wherein values of preset corneal parameters in the non-deformed state of the cornea (26) are varied and the effect of this variation on values of the corneal parameters in the deformed state of the cornea (26) is ascertained for determining the effect of the deformation; determining (S12) the most important corneal parameters for a treatment and/or deformation of the cornea (26) depending on a magnitude of the ascertained effect; adapting (S14) at least one preset fit function as the compensation function of the deformation to the values of the most important corneal parameters; calculating (S16) a deformation-corrected treatment value by means of the compensation function; and providing (S18) the deformation-corrected treatment value for the treatment apparatus (10).

### FIELD

The present invention relates to a method for providing control data for a laser of a treatment apparatus for the correction of a cornea. In addition, the invention relates to a control device for performing the method, to a treatment apparatus with at least one eye surgical laser and at least one control device, to a computer program and to a computer-readable medium.

### BACKGROUND

Treatment apparatuses and methods for controlling lasers for correcting an optical visual disorder of a cornea are known in the prior art. Therein, a pulsed laser and a beam focusing device can for example be formed such that laser beam pulses effect an optical breakthrough in a focus situated within the tissue of the cornea to separate a lenticule from the cornea for correcting the cornea. In the treatment with a treatment apparatus, for example for separating a lenticule, the eye is usually fixed by one or more contact elements of the treatment apparatus. Herein, the contact element is a rigid element, for example a plano-concave lens, which is put onto the eye, in particular onto the cornea, in order that the eye is not moved in the treatment. However, it is disadvantageous in such a contact element that a shape of the cornea changes by the contact element, in particular is compressed. Hereby, the shape of the lenticule to be separated can also change, whereby an originally planned treatment can be erroneous.

Furthermore, the determination, which geometry the lenticule to be removed is to have, is usually performed according to known standard methods, wherein a refractive power to be corrected or a diopter value is for example preset hereto, by means of which the lenticule to be removed can then be determined. In particular the “collapse” or closure of the cornea after removing the lenticule herein results in the desired correction. However, in the determination of the correction of the cornea, in particular in a refractive power correction, which is performed according to standard methods, slight deviations from the actually planned result can occur since an idealized cornea is assumed upon closing the cornea.

The above mentioned deformation effects of the cornea, in particular due to the contact element or by the not exactly modelled closure of the cornea after removing the lenticule, can result in undesired deviations of the treatment result upon a cumulation of these errors. Therefore, one strives to consider and compensate for these effects in advance, wherein the determination of this compensation is often very complicated and time consuming.

### SUMMARY

Therefore, the invention is based on the object to simplify a compensation for deformation effects of the cornea.

This object is solved by the method according to the invention, the apparatuses according to the invention, the computer program according to the invention as well as the computer-readable medium according to the invention. Advantageous configurations with convenient developments of the invention are specified in the respective dependent claims, wherein advantageous configurations of the method are to be regarded as advantageous configurations of the treatment apparatus, of the control device, of the computer program and of the computer-readable medium and vice versa.

A first aspect of the invention relates to a method for providing control data for a laser of a treatment apparatus for the correction of a cornea of a human or animal eye, wherein the method comprises the following steps performed by at least one control device. Therein, an appliance, an appliance component or an appliance group is understood by a control device, which is configured for receiving and evaluating signals as well as for providing, for example generating, control data. For example, the control device can be configured as a control chip, computer program, computer program product or control appliance. In the method, ascertaining an effect of a deformation of the cornea on preset corneal parameters by means of a corneal deformation model is effected by the control device, wherein the cornea can be modeled in a deformed and non-deformed state by the corneal deformation model, wherein values of multiple preset corneal parameters in the non-deformed state of the cornea are varied and the effect of this variation on values of the corneal parameters in the deformed state of the cornea is ascertained for determining the effect of the deformation. Subsequently, determination of the most important corneal parameters for a treatment and/or deformation of the cornea depending on a magnitude of the ascertained effect, adaptation of one or multiple respectively preset fit functions to the values of the most important corneal parameters are effected, wherein the one adapted fit function provides a compensation function for compensating for the deformation or the multiple adapted fit functions are composed to the compensation function. Then, calculating a deformation-corrected treatment value by means of the compensation function and preoperative values of the most important corneal parameters and providing the deformation-corrected treatment value as control data for the treatment apparatus are effected.

In other words, it is first modeled how corneal parameters change by the deformation of the cornea, wherein this can be calculated or simulated by means of the corneal deformation model. The corneal deformation model allows modeling the cornea in a deformed and a non-deformed state. Thus, the deformation by a contact element and/or the closure of the cornea after removing the lenticule and the deformation associated therewith by collapse of the residual corneal layers can for example be modelled. This means that a value (corneal value) of a corneal parameter, for example a radius of curvature of the cornea, in the non-deformed state can be preset and it can be ascertained by means of the corneal deformation model, which effect the deformation of the cornea has on this and the values of the further corneal parameters and thus on a planned treatment result.

Corneal parameters can for example include geometries of the cornea and/or of the lenticule to be separated, which can change before and after the deformation. The deformation or the degree of the deformation can be predetermined or preset, for example by a known radius of curvature of a contact element. For example, the contact element can have a plano-concave shape, a plano-parallel shape or a convex-concave shape. Ascertaining the effect of the deformation of the cornea on the preset corneal parameters can for example be effected in the form of a table, in which a preset corneal parameter, preferably multiple corneal parameters, are varied, wherein the respective values of the further corneal parameters are ascertained for the deformed state in the cornea by the corneal deformation model and stored for each variation, preferably in a proportion, how the respective values in the deformed and non-deformed state vary to each other.

For example, a corneal parameter can be the radius of curvature of the anterior corneal surface, for which a value, for example 7 mm, is first assumed. With the corneal deformation model, it can then be determined how the further corneal parameters, for example the radius of curvature of the anterior interface of the lenticule, change upon a deformation of the cornea if the radius of curvature of 7 mm of the anterior corneal surface is assumed as an initial point. Next, the radius of curvature of the anterior corneal surface can be varied, which means that a second value, for example 8 mm, is assumed, and subsequently the corresponding values can again be ascertained and stored. This variation can preferably be performed for multiple values, in particular a preset range of values, to determine the effect of the deformation of the preset corneal parameters.

As the corneal deformation model, each model can be used, which can describe the cornea and in particular the corneal parameters in the deformed and non-deformed state, wherein the cornea is preferably described as a volume body as the corneal deformation model, which is deformed based on the Euler-Bernoulli beam theory, to ascertain the respective corneal parameters and the effects on the corneal values. A corneal deformation model, which is based on the Euler-Bernoulli beam theory, has proven to be particularly suitable for reproducing these deformation effects. The control device, which is provided for determining the effect of the deformation, can belong to the treatment apparatus or be a control device separate from the treatment apparatus.

Subsequently, it can be determined from the ascertained effect of the deformation, which corneal parameters have the greatest influence on a preset treatment and/or preset deformation of the cornea, whereby the most important corneal parameters can be ascertained. This means that a ranking list can be created based on the deviation determined by the corneal deformation model, wherein the most important corneal parameters can be different according to treatment and/or type of the deformation. Thus, for example with a deformation, which is caused by a contact element, an anterior corneal surface and/or an anterior interface of the lenticule can show the greatest effect on the further corneal parameters. Preferably, at least the two most important corneal parameters can be ascertained.

Furthermore, one or more fit functions can be adapted to the values of the most important corneal parameters. In other words, a single fit function can for example be fitted to the values of the respectively most important corneal parameters or an own fit function can respectively be used for the most important corneal parameters, wherein the respective fit functions can then subsequently be composed to a compensation function. Hereto, a polynomial function can preferably be used as the fit function, in particular a second order polynomial.

The one or the multiple fit functions can then be provided to the treatment apparatus as a compensation function for compensating for the deformation, wherein preoperative values of the corneal parameters, which are derived from predetermined examination data, can be substituted into the compensation function to obtain deformation-corrected treatment values for respective corneal parameters. Preferably, this compensation function is provided to the control device of the treatment apparatus such that the treatment apparatus only requires the compensation function and the ascertainment of the effect of the deformation and the determination of the most important corneal parameters can be performed only once, in particular on a control device separate from the treatment apparatus.

This means that a treatment planning for an individual cornea can be performed at the treatment apparatus after ascertaining the compensation function, in that one or more preoperative values of the most important corneal parameters, which a cornea to be treated actually has, can first be determined. The determination of the preoperative values can be performed according to known approaches, wherein these preoperative values can then be substituted into the compensation function to calculate a deformation-corrected treatment value of a further corneal parameter to be achieved.

This ascertainment of the deformation-corrected treatment value does not have to be performed directly subsequent to the determination of the compensation function, but can occur at any time thereafter. The preoperative values of the most important corneal parameters can for example be ascertained from predetermined examination data, wherein the preoperative values are preferably those, which have been varied in the deformed and non-deformed state for ascertaining the effect and for which the greatest ascertained effect for the treatment to be performed and/or the deformation of the cornea has been determined. Finally, control data, which has the deformation-corrected treatment value, can be provided to the treatment apparatus for controlling the laser. The control data can for example be determined and provided, respectively, for ablative methods, photodisruptive methods, in particular for a lenticule extraction, crosslinking methods of the cornea (Crosslinking; CXL) and/or a method for laser-induced refractive index change (LIRIC).

By the compensation function, thus, a simultaneous compensation, in particular also for cross-effects, which occur upon the deformation of the cornea, can take place for multiple corneal parameters, preferably the most important corneal parameters.

Corneal parameters for example include a radius of curvature of an anterior corneal surface and/or an optical distance between the anterior corneal surface and a posterior corneal surface and/or a thickness of the cornea and/or a radial distance from a limbus to a center of the cornea and/or an optical distance between the anterior corneal surface and an anterior interface of a lenticule to be separated and/or a radius of the anterior interface of the lenticule to be separated and/or a transition zone and/or a thickness of the lenticule and/or a planned refractive power correction and/or a radius of curvature of a contact element and/or a relative thickness of the cornea and/or an incision angle of an incision cut.

By this aspect of the invention, the advantage arises that deformation effects can be easily compensated for and thus better results can be achieved in the treatment. Furthermore, an expensive and complex calculation or simulation, which describes a change of the coordinate system upon the deformation, does not have to be individually performed for each patient, but a one-time determination of the compensation function can take place, which can be used for each treatment apparatus for compensating for the deformation.

The invention also includes forms of configuration, by which additional advantages arise.

A form of configuration provides that the corneal deformation model is based on the Euler-Bernoulli beam theory. In other words, the cornea can be described as a volume body, which deforms based on the Euler-Bernoulli beam theory, to describe the deformed cornea. The Euler-Bernoulli beam theory describes an elastic bending of a body, wherein it is assumed that multiple central corneal surfaces are arranged between an anterior corneal surface and a posterior corneal surface, which constitute the volume body. According to the Euler-Bernoulli beam theory, one of the central corneal surfaces is a neutral corneal surface or neutral membrane, the surface of which remains constant upon the deformation, wherein the further central corneal surfaces can be described depending on the neutral corneal surface. In modeling the deformation by the contact element, the central corneal surfaces below the neutral corneal surface can for example be compressed and those above are stretched. In modeling the closure of the cornea after removing the lenticule, the corneal surfaces, which are situated above the lenticule, can be stretched. Based on the Euler-Bernoulli beam theory, it can be mathematically calculated how the central corneal surfaces change upon an elastic deformation, in particular in relation to the neutral corneal surface. The use of the Euler-Bernoulli beam theory as the corneal deformation model has proven to be particularly suitable since it particularly exactly describes the deformation of the cornea. Thus, improved corneal values for the respective corneal parameters can also be modeled, from which the compensation function can be ascertained.

Preferably, it is provided that the values of the preset corneal parameters are varied within respectively preset ranges of values for determining the effect of the deformation, wherein the ranges of values comprise respective default values of the respective corneal parameter. In other words, a value of a respective corneal parameter can be varied within a range of values, which comprises default values, for determining the effect of the deformation, wherein the default values can for example be known from a patient collective. Thus, only values for the respective corneal parameter can preferably be taken into account, which usually occurs in a patient's eye. Particularly preferably, not all of the values from this range of values are examined by means of the corneal deformation model, but only a preset number of supporting points, which allows a sufficiently exact determination of the compensation for the deformation. By this form of configuration, an effort of modeling the deformation can be reduced.

Particularly preferably, it is provided that the preset fit function is a polynomial function, in particular a second order polynomial. Thus, only a finite number of values can for example be determined by the corneal deformation model for a corneal parameter, for example five values, which are in the range of values of the default values. The values, which are between these points of intersection, can then for example be ascertained in that the present values are fitted by means of a fit function, wherein a second order polynomial has here proven to be particularly suitable. In particular, the compensation function can be composed by means of multiple polynomial functions, wherein the polynomial functions can be fitted for each of the most important corneal parameters. Thus, multiple corneal parameters can be simultaneously compensated for, in particular at the same time, in the compensation function, which is composed of multiple polynomial functions, whereby cross-effects between the different corneal parameters can also be taken into account. A second order polynomial can for example be of the form z(x)=ax²+bx+c, wherein z(x) can be a treatment value of a corneal parameter, which is to be deformation-corrected, and x can be a value of one of the corneal parameters ascertained as important, which is to be substituted into the compensation function as a preoperative value, wherein a, b and c are coefficients in this case, which are obtained from the corneal deformation model by the adaptation to the values of the most important corneal parameters. Alternatively, the polynomial function can be of the form z(x, y)=ax²+b*xy+cy²+dx+ey+f, wherein z (x, y) can be the treatment value to be achieved, which is to be deformation-corrected by the compensation function, x can be a preoperative value of a first important corneal parameter, and y can be a preoperative value of a second important corneal parameter, and a, b, c, d and f can be the coefficients, which have been ascertained from the corneal deformation model from the adaptation of the fit functions to the most important corneal parameters.

Preferably, it is provided that the adapted fit functions of the most important corneal parameters are multiplied by or summed with each other for the compensation function. In other words, with multiple important corneal parameters, multiple fit functions can be adapted to the respective corneal parameters. In order to then obtain the compensation function from the respective fit functions, they can be combined with each other in that a multiplication or summation is performed. For example, z(x) can be a fit function of a first corneal parameter, and z(y) can be the fit function of a second parameter, wherein the compensation function can be z(x, y)=z(x) z(y) or z(x)+z(y).

In an advantageous form of configuration, it is provided that a planned refractive power correction and/or a planned lenticule diameter are adapted by the compensation function. In other words, anterior and posterior interfaces of a lenticule can be determined by means of a planned refractive power correction, thus a diopter value, which is to be compensated for. This planned refractive power correction can be scaled by means of a correction value, which is obtained from the compensation function, or be adapted by means of a differential amount, or the compensation function can provide a global value, which is used as a deformation-corrected refractive power correction. Alternatively or additionally, a lenticule diameter can be planned, in particular a diameter of an optical zone and a magnitude of the correction, respectively, for the correction of the cornea. By means of the compensation function, the planned lenticule diameter can then be adapted via a scaling or a differential amount, to obtain the deformation correction. Alternatively, a global value for the planned lenticule diameter can be calculated from the compensation function. By this form of configuration, the advantage arises that a user, who plans a refractive power correction and/or a lenticule diameter, can simply adapt it by means of the compensation function to compensate for the deformation.

Preferably, it is provided that a deformation of the cornea, which is generated by a contact element, is compensated for by means of the compensation function, and/or wherein a deformation of the cornea, which is generated upon closing the cornea after removing a lenticule from the cornea, is compensated for by means of the compensation function. These two deformations represent the most frequent cause of an erroneous treatment due to deformation effects, wherein they can be compensated for with the aid of the compensation function.

A second aspect of the present invention relates to a control device, which is configured to perform the above described method. The above cited advantages arise. For example, the control device can be configured as a control chip, control appliance or application program (“app”). The control device can preferably comprise a processor device and/or a data storage. By a processor device, an appliance or an appliance component for electronic data processing is understood. For example, the processor device can comprise at least one microcontroller and/or at least one microprocessor. Preferably, a program code for performing the method according to the invention can be stored on the optional data storage. Then, the program code can be adapted, upon execution by the processor device, to cause the control device to perform one of the above described embodiments of the method according to the invention. Furthermore, the control device can comprise multiple control units, in particular a first control unit, which is formed for calculating the look-up table and can be formed independently of the treatment apparatus, and a second control unit, which is formed for determining the deformation-corrected corneal value to be achieved by means of the look-up table and providing the control data, wherein the second control unit is preferably arranged in the treatment apparatus. In other words, the control unit in the treatment apparatus can preferably only include the look-up table, which has been provided by the first control unit.

A third aspect of the present invention relates to a treatment apparatus with at least one eye surgical laser for the separation of a lenticule with predefined interfaces from a human or animal eye by means of optical breakthroughs and/or ablation, and with at least one control device for the laser or lasers, which is formed to execute the steps of the method according to the first aspect of the invention. Preferably, the above mentioned two control units are provided for the treatment apparatus.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 Megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is ensured in the generation of the interfaces. In particular, the range between 700 nm and 780 nm can also be selected as the wavelength range.

In further advantageous configurations of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

Further features and the advantages thereof can be taken from the descriptions of the first inventive aspect, wherein advantageous configurations of each inventive aspect are to be regarded as advantageous configurations of the respectively other inventive aspect.

A fourth aspect of the invention relates to a computer program including commands, which cause the control device according to the second inventive aspect to execute the method steps according to the first inventive aspect.

A fifth aspect of the invention relates to a computer-readable medium, on which the computer program according to the fourth inventive aspect is stored. Further features and the advantages thereof can be taken from the descriptions of the first to fourth inventive aspects, wherein advantageous configurations of each inventive aspect are to be regarded as advantageous configurations of the respectively other inventive aspect.

### BRIEF DESCRIPTION OF THE DRAWINGS

Further features of the invention are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 depicts a schematic representation of a treatment apparatus according to an exemplary embodiment.

FIG. 2 depicts a schematic method diagram for providing control data according to an exemplary embodiment.

FIG. 3a depicts a schematically illustrated cornea of the corneal deformation model in the non-deformed state.

FIG. 3b depicts the cornea of the corneal deformation model deformed by a contact element.

FIG. 4a depicts a schematically illustrated cornea of the corneal deformation model in the non-deformed state before removal of a lenticule.

FIG. 4b depicts the deformed cornea of the corneal deformation model after closing the lenticule.

FIG. 5a depicts an exemplary representation of a first varied corneal parameter.

FIG. 5b depicts an exemplary representation of a second varied corneal parameter.

In the figures, identical or functionally identical elements are provided with the same reference characters.

### DETAILED DESCRIPTION

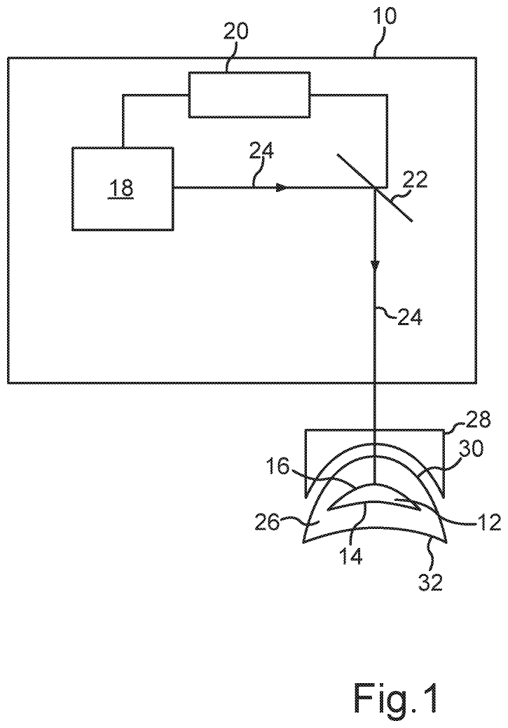

FIG. 1 shows a schematic representation of a treatment apparatus 10 with an eye surgical laser 18 for the separation of a lenticule 12 defined by control data from a cornea 26 by means of photodisruption and/or ablation, wherein the cornea 26 is bounded by an anterior corneal surface 30 and a posterior corneal surface 32 in the direction of an optical axis. For separating the lenticule 12, a posterior interface 14 and an anterior interface 16 of the lenticule 12 are preset in the control data, on which a cavitation bubble path for separating the lenticule 12 from the cornea 26 can be generated. One recognizes that a control device 20 for the laser 18 can be formed besides the laser 18 such that it can emit pulsed laser pulses for example in a predefined pattern for generating the interfaces 14, 16. Alternatively, the control device 20 can be a control device 20 external with respect to the treatment apparatus 10.

Furthermore, FIG. 1 shows that the laser beam 24 generated by the laser 18 is deflected towards the cornea 26 by means of a beam device 22, namely a beam deflecting device such as for example a rotation scanner. The beam deflecting device 22 is also controlled by the control device 20 to generate the interfaces 14, 16, preferably also incisions or cuts, along preset incision courses.

The illustrated laser 18 can preferably be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. Optionally, the control device 20 additionally comprises a storage device (not illustrated) for at least temporary storage of at least one control dataset, wherein the control dataset or datasets include(s) control data for positioning and/or for focusing individual laser pulses in the cornea. The position data and/or focusing data of the individual laser pulses, that is the lenticule geometry of the lenticule 12 to be separated, are generated based on predetermined control data, in particular from a previously measured topography and/or pachymetry and/or the morphology of the cornea or of the optical visual disorder correction to be generated.

For determining the visual disorder data, which can for example indicate a value in diopters, suitable examination data for describing the visual disorder can be received by the control device 20 from a data server or the examination data can be directly input into the control device 20.

Further, a contact element 28 can be provided, which can belong to the treatment apparatus 10. Alternatively, the contact element 28 can also be provided separately from the treatment apparatus 10. The contact element 28, which can also be referred to as patient interface or fixing system, serves to fix the eye or the cornea 26 for the treatment. Hereto, the contact element 28 can comprise a plano-concave lens, which is adapted to the cornea 26 for fixing. By fixing by means of the contact element 28, however, it can occur that the cornea 26 deforms and thus the geometry of the lenticule 12 does no longer have the originally planned dimensions. Therefore, it can occur that a planned refractive power value or refractive power value to be corrected for example deviates from an achieved refractive power value after the treatment with the treatment apparatus 10.

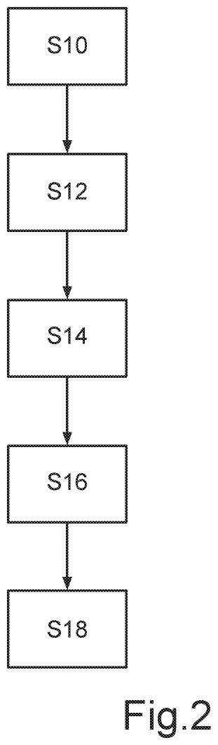

In FIG. 2, a schematic method diagram for providing control data for the laser 18 of the treatment apparatus 10 is illustrated, which can for example be performed by the control device 20. In a step S10, an effect of a deformation of the cornea on preset corneal parameters can first be determined by means of a corneal deformation model, wherein the corneal deformation model can describe the cornea 26 as a volume body, and which is preferably based on the Euler-Bernoulli beam theory. Thus, the cornea 26 can be modeled in a deformed and non-deformed state, wherein a value of at least one corneal parameter in the non-deformed state of the cornea is varied and an effect of this variation on values of the further corneal parameters in the deformed state is ascertained for determining the effect of the deformation.

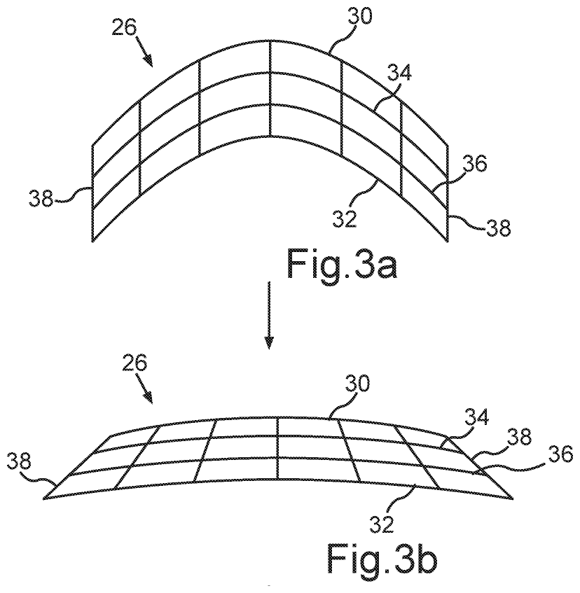

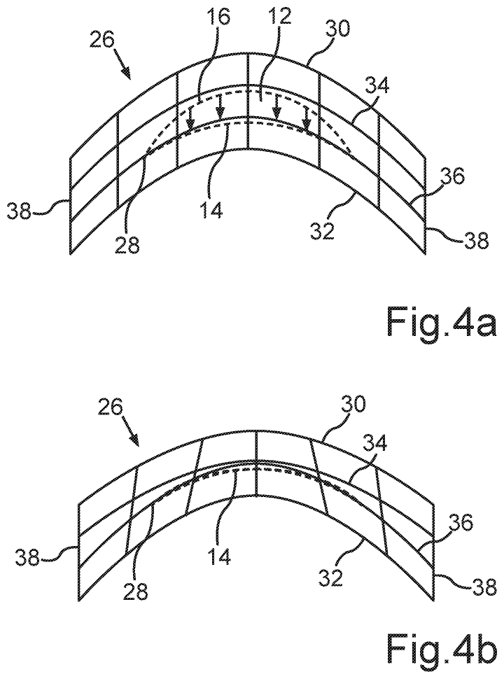

For illustrating the corneal deformation model, the deformation of the volume body of the cornea 26 is shown for the deformation by the contact element 28 in FIGS. 3a and 3b and is shown for the deformation, which occurs in closing the cornea 26 after removing the lenticule 12 in FIGS. 4a and 4b.

Herein, FIG. 3a for example shows the volume body of the cornea 26 in a free state before the deformation by the contact element 28, which is not illustrated in this figure. Therein, the volume body can be bounded by the anterior corneal surface 30 and the posterior corneal surface 32 in the direction of the optical axis and by lateral interfaces 38 in radial direction (lateral). Herein, the anterior corneal surface 30 and the posterior corneal surface 32 can be provided as ellipsoids, wherein a two-dimensional cross-section through the volume body is shown in this figure for illustration and the volume body can be present in a three-dimensional shape, in particular rotationally symmetrical. Besides the anterior and posterior corneal surfaces 30, 32, central corneal surfaces 34, 36 of the volume body are also illustrated, wherein a central corneal surface can be provided within the volume body for each position in z-direction (direction of the optical axis), which is not shown here for reasons of clarity. One of the central corneal surfaces, for example the central corneal surface 36, can be a neutral corneal surface or neutral membrane, which has the same surface before and after the deformation according to the Euler-Bernoulli beam theory, which is taken into account in modeling the cornea 26 based on the corneal deformation model. Preferably, a respectively central corneal surface 34 can be described in relation to this neutral corneal surface in the corneal deformation model.

Thus, a radius of curvature of a respectively central corneal surface 34 can preferably be described by means of the corneal deformation model according to the formula

$$\frac{1}{{r}_{cent , pre}} = ( \frac{q}{{r}_{ca}} + \frac{1 - q}{{r}_{cp}} )$$

wherein it provides the radius of curvature of the central corneal surface 34 before the deformation (r_{cent,pre}). Therein, r_{ca} describes the radius of curvature of the anterior corneal surface 30 and rip describes the radius of curvature of the posterior corneal surface 32. The variable q describes a relative position of the central corneal surface 34 to the neutral corneal surface 36, wherein q can take a value between 0 and 1.

In similar manner, a position in z-direction, which is dependent on the radial position, can also be described to the radius of curvature, wherein the z-direction extends in the direction of the optical axis. It can be described for the respective central corneal surface 34 with the formula

$${𝓏}_{cent , pre} ( {r}_{x} ) = ( q - 1 ) {d}_{cc} - \frac{{r}_{x}^{2}}{2} ( \frac{q}{{r}_{ca}} + \frac{1 - q}{{r}_{cp}} )$$

wherein r_{X} describes a radial position starting from the center of the cornea 26 and dcc describes a central thickness of the cornea 26 at the highest point or inflection point of the cornea 26.

Upon the deformation of the cornea 26 by the contact element 28, it can be provided in the corneal deformation model that the radius of curvature of the anterior corneal surface 30 is adapted to a radius of curvature of the contact element 28. This situation is for example illustrated in FIG. 3b, wherein the contact element 28 is not shown here for reasons of clarity. It is seen that the anterior corneal surface 30 is impressed and thus also the central corneal surfaces 34 and 36. However, according to the Euler-Bernoulli beam theory, it further remains considered that the neutral corneal surface 36 has the same surface as before the deformation. In this deformation, it is assumed that the volume body can freely deform and is not bounded towards the sides.

In FIG. 4a, the cornea 26 is illustrated in a non-deformed state before the removal of the lenticule 12. Here too, the cornea 26 can be modeled as a volume body, which is formed of respective central corneal surfaces 34, 36, wherein the anterior interface 16 of the lenticule is pressed onto the posterior interface 14 of the lenticule 12 for determination of the deformed cornea in the corneal deformation model, whereby they change the curvatures of the corneal surfaces 30, 34 situated above. Therein, the corneal deformation model is based on the same principles and formulas as already described to the FIGS. 3a and 3b.

In the deformation of the cornea 26 by closing the area of the lenticule 12, it can be provided in the corneal deformation model that the radius of curvature of the anterior interface 16 is adapted to a radius of curvature of the anterior interface 14 such that the cornea 26 according to FIG. 4b results. Herein, the anterior interface 16 can move downwards to the posterior interface 14, wherein the corneal surfaces situated above the anterior interface are thus also adapted, in particular the neutral corneal surface 34 and the anterior corneal surface 30.

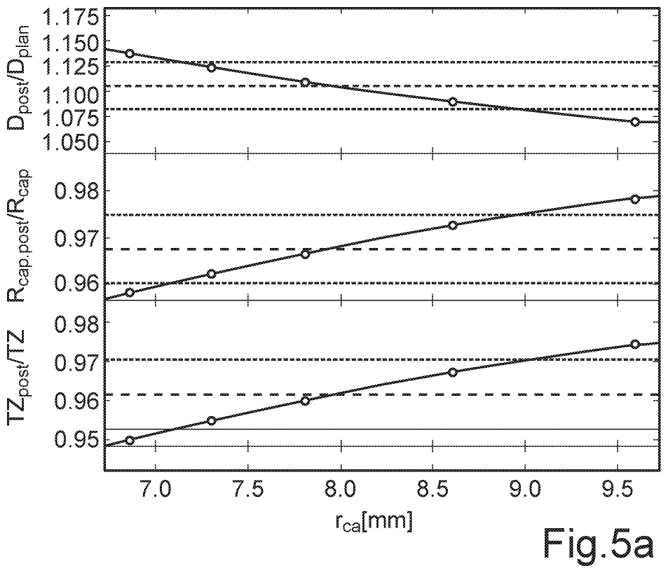

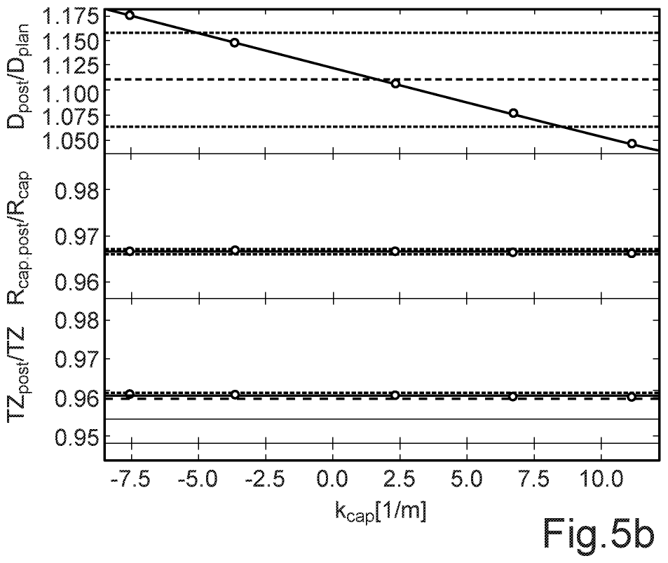

In FIGS. 5a and 5b, exemplary variations of corneal parameters and the effect of them on further corneal parameters in the deformed state of the cornea are illustrated as they can be performed in the method step S10. Therein, effects on the corneal parameters, which can be induced by a deformation of the cornea 26 by the contact element 28, are shown in both figures FIGS. 5a and 5b.

On the x-axis of FIG. 5a, the corneal parameter r_{ca} is represented, which represents a radius of curvature of the anterior corneal surface 30. This corneal parameter r_{ca} is varied within a preset range of values, which preferably comprises default values of the corneal parameter from a patient collective, and the effect of this variation on further corneal parameters, which are represented on the y-axis of FIG. 5a, is ascertained. In this example, the further corneal parameters are a refractive power, in particular a ratio of a planned refractive power correction Dₚₗₐₙ and the refractive power correction Dₚₒₛₜ ascertained by the corneal deformation model, a ratio of the planned radius of the anterior interface 16 ascertained by the corneal deformation model (R_{cap}) and a ratio of the planned lenticule diameter ascertained by the corneal deformation model (including the transition zone TZ). Besides these exemplarily shown corneal parameters, effects on further corneal parameters can also be ascertained in the corneal deformation model, such as for example an optical distance between the anterior corneal surface and a posterior corneal surface, a thickness of the cornea, a radial distance from a limbus to a center of the cornea, an optical distance between the anterior corneal surface and an anterior interface of a lenticule to be separated, a thickness of the lenticule, a radius of curvature of the contact element, a relative thickness of the cornea and/or an incision angle of an incision cut.

For determining the graphic shown in FIG. 5a, it can for example be proceeded as follows by means of the corneal deformation model: As a first preset corneal parameter, the radius of curvature of the anterior corneal surface r_{ca} can be selected, wherein it has to have a radius of curvature of 7 mm in the non-deformed state as a first value. By the corneal deformation model, the cornea, which has a radius of curvature of 7 mm, is deformed, wherein the effect, which the deformation of the cornea, which has a radius of curvature of 7 mm, has on the further corneal parameters, which are plotted on the y-axis, is ascertained. Subsequently, the corneal parameter r_{ca} can be varied, which means that a radius of curvature of 7.5 mm in the non-deformed state is next assumed and the cornea is deformed by means of the corneal deformation model in the same manner as previously described, and the effect on the further corneal parameters is ascertained. This variation can then be repeated until a sufficient number of values is ascertained. When the entire range of values of the radius of curvature of the anterior corneal surface r_{ca} has been determined, thus, it can be comprehended with known radius of curvature of a real cornea how a planned refractive power correction Dₚₗₐₙ for example changes to Dₚₒₛₜ by the deformation of the cornea.

In corresponding manner, further corneal parameters can also be varied except for the radius of curvature of the anterior corneal surface r_{ca} 30, as for example illustrated in FIG. 5b. FIG. 5b is substantially identically configured as FIG. 5a, wherein an optical distance between the anterior corneal surface 30 and the anterior interface of the lenticule 12 to be separated, which is here denoted by k_{cap}, is illustrated as the corneal parameter, which is varied, in FIG. 5b. This means that the corneal parameter k_{cap} is varied in FIG. 5b and the effect of the deformation of the cornea on the further corneal parameters, which are the same as in FIG. 5a in this case, is stored for the respective value of k_{cap} in the non-deformed state.

Returning to the method diagram of FIG. 2, after determining the effect of the deformation in step S10 (which are illustrated in FIGS. 5a and 5b), it can be determined in a step S12, which are the most important corneal parameters for a treatment and/or deformation of the cornea 26 in that those corneal parameters are determined, which have the greatest effect by the deformation. In this example, the deformation of the cornea can be a deformation by the contact element 28, and the treatment can be a refractive power correction of the cornea 26. From the effects of the deformation ascertained in step S10, for example from multiple tables or graphs, which can be similarly constructed as FIGS. 5a, 5b, wherein further corneal parameters not shown here are additionally varied, it can then be determined, which initially assumed corneal parameters cause the greatest effect on the refractive power correction by the deformation of the cornea 26. In this example, the corneal parameters r_{ca} and k_{cap} shown in FIGS. 5a and 5b can have the greatest effect of all of the corneal parameters on the refractive power correction Dₚₒₛₜ/Dₚₗₐₙ, wherein more than two corneal parameters can also be determined. For reasons of clarity, the example is subsequently continued with two corneal parameters (r_{ca}, k_{cap}).

After determining the most important corneal parameters for the treatment and/or deformation, one or multiple respectively preset fit functions can be adapted to the values of the ascertained most important corneal parameters in a step S14, to determine a compensation function, which can be provided for compensating for the deformation, in particular the refractive power correction. As illustrated in FIGS. 5a and 5b, only a finite number of values is preferably ascertained, in this example five values or supporting points. The respective fit functions can then be adapted to these values, wherein a polynomial function, in particular a second order polynomial, can preferably be used as the fit function, to obtain the values of the entire parameter range of the respective corneal parameter. In other words, the first polynomial function can be fitted to the values of Dₚₒₛₜ/Dₚₗₐₙ of FIG. 5a and a second polynomial function can be fitted to the corresponding values of FIG. 5b, wherein the respective polynomial function can be identical or different, or a single fit function can be adapted to both functions, in particular a mixed polynomial with two variables.

In this example, a first polynomial function can have been adapted to FIG. 5a and a second polynomial function can have been adapted to FIG. 5b for the refractive power correction Dₚₒₛₜ/Dₚₗₐₙ, wherein the two polynomial functions can be composed to a compensation function. In particular, the respective polynomial functions/fit functions can be combined as a product or sum in the compensation function, wherein the type of the operation can depend on the respective corneal parameter.

This compensation function can then be provided to the treatment apparatus 10, in particular to the control device 20, for compensating for the deformation to perform a deformation-corrected refractive power correction.

Thus, a deformation-corrected treatment value can be calculated by means of the compensation function in a step S16, in that preoperative values of the most important corneal parameters are substituted into the compensation function and thus the deformation-corrected treatment value is generated. In this example, the deformation-corrected treatment value is the refractive power correction, which is to be ascertained by the compensation function. In order to correct the originally planned refractive power correction for the deformation to be expected, the preoperative values of the most important corneal parameters, which are r_{ca} and k_{cap} in this example, can be ascertained from predetermined examination data, wherein they can be substituted into the ascertained compensation function. Thus, both the influence of the corneal parameter r_{ca} and k_{cap} upon deformation of the cornea 26 on the refractive power correction can be compensated for by the compensation function, in particular at the same time, which provides an improved deformation correction since the most important corneal parameters are taken into account in the compensation function. In corresponding manner, a compensation function with the most important corneal parameters can also be ascertained for the further corneal parameters R_{cap}, TZ and further corneal parameters, wherein the deformation can then be compensated for also for these corneal parameters in corresponding manner.

Finally, the thus obtained deformation-corrected treatment values can be provided as control data for the treatment apparatus 10, in particular the control device 12, in a step S18.

Overall, the examples show how a simple and fast compensation for deformation effects can be achieved by means of the compensation function.

1. A method for providing control data for a laser of a treatment apparatus for the correction of a cornea of a human or animal eye, wherein the method comprises the following steps performed by at least one control device:
ascertaining an effect of a deformation of the cornea on preset corneal parameters by means of a corneal deformation model, wherein the cornea can be modeled in a deformed state and a non-deformed state by the corneal deformation model, wherein values of multiple preset corneal parameters in the non-deformed state of the cornea are varied and the effect of this variation on values of the corneal parameters in the deformed state of the cornea is ascertained for determining the effect of the deformation;
determining the most important corneal parameters for a treatment and/or deformation of the cornea depending on a magnitude of the ascertained effect;
adapting one or multiple respectively preset fit functions to the values of the most important corneal parameters, wherein the one adapted fit function provides a compensation function for compensating for the deformation or the multiple adapted fit functions are composed to the compensation function;
calculating a deformation-corrected treatment value by means of the compensation function and preoperative values of the most important corneal parameters; and
providing the deformation-corrected treatment value as control data for the treatment apparatus.

2. The method according to claim 1, wherein the corneal deformation model is based on the Euler-Bernoulli beam theory.

3. The method according to claim 1, wherein the values of the preset corneal parameters are varied within respectively preset ranges of values for determining the effect of the deformation, wherein the ranges of values comprise respective default values of the respective corneal parameter.

4. The method according to claim 1, wherein the preset fit function is a polynomial function, in particular a second order polynomial.

5. The method according to claim 1, wherein the adapted fit functions of the most important corneal parameters are multiplied by or summed with each other for the compensation function.

6. The method according to claim 1, wherein a planned refractive power correction and/or a planned lenticule diameter are adapted by the compensation function.

7. The method according to claim 1, wherein a deformation of the cornea, which is generated by a contact element, is compensated for by means of the compensation function, and/or wherein a deformation of the cornea, which is generated upon closing the cornea after removal of a lenticule from the cornea, is compensated for by means of the compensation function.

8. A control device that is formed to perform a method according to claim 1.

9. A treatment apparatus with at least one eye surgical laser for the separation of a lenticule with predefined interfaces from a human or animal eye by cavitation bubbles and with at least one control device according to claim 8.

10. The treatment apparatus according to claim 9, wherein the at least one eye surgical laser is suitable to emit laser pulses in a wavelength range between 300 nm and 1400 nm, at a respective pulse duration between 1 fs and 1 ns, and a repetition frequency of greater than 10 kHz.

11. The treatment apparatus according to claim 9, wherein the control device
comprises at least one storage device for at least temporary storage of at least one control dataset, wherein the control dataset or datasets include(s) control data for positioning and/or for focusing individual laser pulses in the cornea; and
includes at least one beam device for beam guidance and/or beam shaping and/or beam deflection and/or beam focusing of a laser beam of the laser.

12. A computer program including commands that cause the control device according to claim 8 to execute the method.

13. A non-transitory computer-readable medium, on which the computer program according to claim 12 is stored.
